(19) United States
(12) Patent Application Publication
(10) Pub. No.: US 2013/0316991 A1
(43) Pub. Date: Nov. 28, 2013

(54) Biologically active supplements and process of its production

(71) Applicant: GAMAVETFARM LTD, (US)

(72) Inventors: Alexandr Naumovich NAROVLYANSKIJ, Moskow (RU); Aleksandr Vasil'evich PRONIN, Moskow (RU); Alexandr Vladimirovich SANIN, Moskow (RU); Aleksej Yur'evich SVIRIDOV, Moskow (RU); Mariya Valentinovna STARIKOVA, Moskow (RU); Alexandr Vladimirovich KHOMICH, Moskow (RU)

(73) Assignee: GAMAVETFARM LTD, Moscow (RU)

(21) Appl. No.: 13/727,725

(22) Filed: Dec. 27, 2012

Related U.S. Application Data
(63) Continuation of application No. PCT/RU2010/000662, filed on Nov. 11, 2010.

(30) Foreign Application Priority Data
Jun. 29, 2010 (RU) 2010126530
Nov. 11, 2010 (RU) PCT/RU2010/000662

(51) Int. Cl. A61K 31/663; A61K 31/575

(52) U.S. Cl. CPC A61K 31/663; A61K 31/575
USPC 514/171; 514/102

(57) ABSTRACT

The invention relates to dietary and prophylactic biologically active food supplements (BAS), in particular, from raw plant material and methods for their production; it can be used as a food supplement for human and domestic animals to reduce the cholesterol content in blood, stimulation of protein glycosylation and prophylaxis of infectious and cardiovascular diseases.

The technical result of applying the BAS proposed consists in the creation of a drug for reducing the cholesterol content in blood, stimulation of protein glycosylation and prophylaxis of infectious and cardiovascular diseases.

The biologically active supplement contains at least one polyprenyl phosphates or polyprenyl pyrophosphate with isoprene components from 7 to 30 or a mixture of different polyprenyl phosphates and polyprenyl pyrophospates adsorbed on a solid sorbent (sorbite, lactose, and starch) and also beta-sitosterin.

The biologically active supplement is produced by the dissolution of a mixture of polyprenyl phosphates and/or polyprenyl pyrophospates in an organic solvent, addition of a sorbent to the solution obtained and subsequent mixing and distillation of the solvent. Beta-sitosterin can be also added along with the sorbent.

The invention relates to dietary and prophylactic biologically active food supplements (BAS) from raw plant material on the basis of polyprenyl phosphates and polyprenyl pyrophospates and to methods for their production; it may be used as a food supplement for human and domestic animals to reduce the cholesterol content in blood, stimulation of protein glycosylation, and prophylaxis of infectious and cardiovascular diseases.

### BACKGROUND

The possibility of applying polyprenols and their phosphates is limited by the fact that they represent a lipoid mass, which is practically insoluble in water and resisted to drying and grinding. Therefore, they are dissolved in a complex solvent and used only orally or as injections. This significantly complicates storage conditions and limits a sphere of applying BAS by the field of medicine and veterinary.

The extension of the sphere of applying the specified agent requires its production in the form of tablets, capsules or powder suitable for the use as a supplement to food for animals.

Some BAS for the reduction of the cholesterol amount in blood, stimulation of protein glycosylation and prophylaxis of infectious and cardiovascular diseases are unknown to the authors.

### SUMMARY OF THE INVENTION

A technical result of applying the BAS proposed consists in the elaboration of a drug for reducing the cholesterol content in blood, stimulation of protein glycosylation and prophylaxis of infectious and cardiovascular diseases.

This problem can be solved by an application of polyprenols and/or their phosphates to biologically inert carriers.

The technical result specified is achieved by the fact that the BAS for reducing the cholesterol content in blood, stimulation of protein glycosylation and prophylaxis of infectious and cardiovascular diseases contains at least one polyprenyl phosphate or polyprenyl pyrophosphate with 7 to 30 isoprene components or a mixture of different polyprenyl phosphates and polyprenyl pyrophospates adsorbed on a solid sorbent, particularly on sorbite, lactose, and starch.

The technical result specified is also achieved by the fact that the content of polyprenyl phosphates in a mixture of polyprenyl phosphates and polyprenyl pyrophospates amounts to 5-20 wt %.

The technical result specified is also achieved by the fact that the content of polyprenyl phosphates in a mixture of polyprenyl phosphates and polyprenyl pyrophospates amounts to 10 wt %.

The technical result specified is also achieved by the fact that the content of the sorbent amounts to 90.0-99.5 wt %.

The technical result specified is also achieved by the fact that the content of a sorbent amounts to 98 wt %.

The technical result specified is achieved by the fact that the biologically active supplement also contains beta-sitosterin.

The technical result specified is also achieved by the fact that the beta-sitosterol content is 1-10 wt %.

The technical result specified is also achieved by the fact that the beta-sitosterol content is 5 wt %.

The technical result specified is also achieved by the fact that the biologically active supplement is produced by dissolving a mixture of polyprenyl phosphates and/or polyprenyl pyrophospates in an organic solvent, addition of the sorbent to the solution obtained, and subsequent mixing and the solvent distillation.

The technical result specified is also achieved by the fact that beta-sitosterol is added along with the sorbent.

The technical result specified is also achieved by the fact that the distillation of the solvent is performed after mixing.

### DETAILED DESCRIPTION

The BAS is obtained in the following way.

Polyprenyl phosphates, polyprenyl pyrophospates or their mixture are dissolved in an organic solvent and mixed with a sorbent; then, the solvent is distilled out. In the course of the distillation, polyprenols are adsorbed on the sorbent.

The following process can be presented as an example.

20 g of polyprenyl phosphates are dissolved in 200 g of an organic solvent (acetone, hexane, etc.); 980 g of sorbite, lactose or starch are added to the solution obtained. The mixture is carefully mixed, placed to a rotary evaporator, and the solvent is distilled out during an hour. The mass obtained is taken out and placed into a thermostat to dry it at 40° C. during 3-4 h. After drying, the solid mass is powdered using a mill and sifted to obtain a fine homogenous powder ready for the use.

The BAS is added to food or forage for animals or used in the form of tablets and capsules.

Some examples for the action of the BAS proposed are given below.

### DETAILED DESCRIPTION OF THE PREFERRED EMBODIMENTS

### EXAMPLE 1

The powder prepared according to example 1 with the use of starch as a sorbent is added (450 g/t) to the standard food for 10-day-old pigs (100 animals). Young pigs of the control group (100 animals) obtain standard food. After 15 days, the safety of the animals from the experimental group amounted to 98.5-100%; the safety of the control group—97.0-97.8%. Gastroenteric upset was observed in 0-8 young pigs of the experimental group and in 20-22 animals of the control group. Thus, the application of the BAS allows reducing the morbidity and raising the safety of livestock to a great extent.

### EXAMPLE 2

Two groups of 5-10-day-old calves (having 10 animals each) are kept under similar sanitary-hygienic conditions—each group in an individual cage of a closed house. The animals obtained pigswill three times a day during a month. It consisted of milk for the control group and milk with a powder prepared with lactose (PrenoLakt) as a sorbent at 0.125 g per 1 kg of milk. By the end of the experiment, the mean weight gain is 25.6±1.8 kg per a calf in the test group and 21.2±1.2 kg in the control one; in the test group, gastroenteric upsets in animals were absent; in the control group, they were observed in 5 calves. Thus, the application of the BAS allows raising the weight gain and reducing the morbidity of calves.

### EXAMPLE 3

The 28-day-old broiler chickens (140000 animals) receive standard food with a powder prepared on the basis of lactose (PrenoLakt) in a dose of 450 g/t of food during 5 days. In the control group, the chickens receive an ordinary food. By the end of the experiment, the mortality of animals from the test group significantly decreases (0.07-0.08% versus 0.14% in the control). Thus, the application of the BAS permits to reduce the mortality of animals to a great extent.

### EXAMPLE 4

### The Influence of BAS on the Intensity of Protein Synthesis in Macrophages

Mice CBA with a mass of 18-20 g receive BAS one time orally in a dose of 10 mg/animal. In 1 and 7 days, peritoneal macrophages are obtained and investigated using ³H-thymidine labeled amino acids. Table 1 shows that the intensity of protein synthesis significantly increases in 7 days after the BAS administration.

TABLE 1
The influence of BAS on the intensity of protein synthesis in macrophages

| | Count/min per mg of |
|---|---|
| Group | protein |
| Control | 18947 ± 949 |
| 1 day | 18060 ± 776 |
| 7 days | 53391 ± 7351 |

### EXAMPLE 5

### The Influence of BAS on the Production of Glycoproteins (Immunoglobulins)

The BAS containing only polyprenyl phosphates (C1), polyprenyl phosphates and polyprenyl pyrophospates in the 9:1 (C2) and 1:1 (C3) ratios, are introduced orally to mice CBA with a mass of 18-20 g in a dose of 10 or 50 mg/animal three times—a day before the immunization, at the moment of the immunization, and a day after the immunization. The animals are immunized intravenously with 4×10⁸ of sheep's erythrocytes. After 5 days, a blood serum is obtained, and titers of antibodies are determined using 96-well plates. 0.025 ml of physiological solution and 0.025 ml of the serum in different dilutions are poured into each well, and then, 0.025 ml of 1% sheep's erythrocytes is added there. The plate is incubated during 3 h at 37° C. The serum dilution, where the agglutination of erythrocytes is still observed, is considered as an antibody titer.

TABLE 2
The effects of the BAS on antibody titers towards sheep's erythrocytes

Antibody titers (log₂ ± m)

| Group | C1 | C2 | C3 |
|---|---|---|---|
| Control | 5.3 ± 0.9 | 5.3 ± 0.9 | 5.3 ± 0.9 |
| 10 mg | 5.5 ± 1.2 | 5.8 ± 0.5 | 5.9 ± 0.8 |
| 50 mg | 7.0 ± 0.7 p < 0.05 | 7.3 ± 0.8 | 7.2 ± 0.9 |

The BAS in a dose of 50 mg significantly increases the titer of antibodies, the activity of which is related to the presence of glycated immunoglobulins. There are no significant differences between C1, C2 and C3.

### EXAMPLE 6

### The Effect of the BAS on the Cholesterol Content in Blood

A group of 5 volunteers at an age from 42 to 62 years receive 130 mg of BAS on the basis of sorbite with 5% beta-sitosterin (SitoPren) twice every day during 3 months. At the beginning of the experiment (0), in 22 days, and in 3 months after the BAS intake, the contents of cholesterol, high-density lipoproteins (HDL) and low-density lipoproteins (LDL) are analyzed. If before the intake of BAS, the cholesterol content is on the upper normal level, and the LDL content is somewhat higher than the norm, by the end of the experiment, these parameters have lower values at the same HDL level.

TABLE 3
The contents of cholesterol and high-density lipoproteins
(HDL) and low-density lipoproteins (LDL) in blood of
volunteers in different terms after the BAS intake

| | Content (mM/ml) | | | |
|---|---|---|---|---|
| Parameter | Norm | 0 | 22 days | 3 months |
| Cholesterol | 0-6.0 | 6.0 ± 0.4 | 6.13 ± 0.3 | 5.48 ± 0.21 |
| HDL | 1.03-1.55 | 1.54 ± 0.59 | 1.52 ± 0.17 | 1.54 ± 0.36 |
| LDL | 0-4.1 | 4.66 ± 0.78 | 4.5 ± 0.58 | 4.05 ± 0.12 |

1-11. (canceled)

12. A biologically active supplement for lowering the cholesterol content in blood or prophylaxis of cardiovascular diseases comprising at least one polyprenyl phosphate with a number of isoprene components from 7 to 30, at least one polyprenyl pyrophosphate with a number of isoprene components from 7 to 30 or a mixture of polyprenyl phosphates and polyprenyl pyrophospates, said polyprenyl phosphate, said polyprenyl pyrophosphate or said mixture adsorbed on a solid sorbent.

13. The biologically active supplement of claim 12, wherein the biologically active supplement comprises the mixture of polyprenyl phosphates and polyprenyl pyrophospates, and the polyprenyl phosphates comprise 5-20% wt % of the mixture.

14. The biologically active supplement of claim 13, wherein said polyprenyl phosphates comprise 10 wt % of the mixture.

15. The biologically active supplement of claim 12, the sorbent comprising 90.0-99.5 wt % of the biologically active supplement.

16. The biologically active supplement of claim 15, wherein the sorbent comprises 98.0 wt % of the biologically active supplement.

17. The biologically active supplement of claim 15, further comprising beta-sitosterin.

18. The biologically active supplement of claim 17, wherein the beta-sitosterin comprises 1-10 wt % of the biologically active supplement.

19. The biologically active supplement of claim 18, wherein the beta-sitosterin comprises 5 wt % of the biologically active supplement.

20. A process for producing a biologically active supplement comprising polyprenyl phosphates and/or polyprenyl pyrophospates, the process comprising
dissolving a mixture of polyprenyl phosphates and/or polyprenyl pyrophospates in an organic solvent to obtain a solution,
adding a sorbent to the solution, and
distilling the solution comprising the sorbent.

21. The process according to claim 20, wherein beta-sitosterin is added to the solution along with the sorbent.

22. The process of claim 20, comprising mixing the solution comprising the sorbent before the distilling.

23. The process of claim 21, comprising mixing the solution comprising the sorbent and the beta-sitosterin before the distilling.

24. The process of claim 20, wherein the biologically active supplement is a biologically active supplement for lowering the cholesterol content in blood and prophylaxis of cardiovascular diseases according to claim 12.

25. The biologically active supplement of claim 12, wherein the solid sorbent is selected from the group consisting of sorbite, lactose, and starch.

26. A method of lowering the cholesterol content in blood or prophylaxis of cardiovascular diseases comprising administering the biologically active supplement of claim 1 to an animal.

27. The method of claim 26, wherein the biologically active supplement is administered in the form of a tablet or a capsule.

28. The method of claim 26, wherein the biologically active supplement is added to forage for animals.
